(19) United States
(12) Patent Application Publication
(10) Pub. No.: US 2014/0288344 A1
(43) Pub. Date: Sep. 25, 2014

(54) PROCESS FOR THE CONVERSION OF FEEDS OBTAINED FROM RENEWABLE RESOURCES USING A CATALYST COMPRISING A Nu-10 ZEOLITE AND A SILICA-ALUMINA

(71) Applicant: IFP ENERGIES NOUVELLES, Rueil-Malmaison Cedex (FR)

(72) Inventors: Filipe Manuel MARQUES MOTA, Lyon (FR); Christophe BOUCHY, Lyon (FR); Pascal DUCHENE, Vienne (FR); Johan MARTENS, Huldenberg (BE)

(73) Assignee: IFP ENERGIES NOUVELLES, RUEIL-MALMAISON CEDEX (FR)

(21) Appl. No.: 14/219,058

(22) Filed: Mar. 19, 2014

(30) Foreign Application Priority Data
Mar. 21, 2013 (FR) 13/52527

(51) Int. Cl. C07C 5/22

(52) U.S. Cl. CPC C07C 5/222
USPC 585/739

(57) ABSTRACT

The invention concerns a process for the conversion of a paraffinic feed produced from renewable resources, to the exclusion of paraffinic feeds obtained by a process employing a step for upgrading by the Fischer-Tropsch pathway, said process employing a catalyst comprising at least one hydrodehydrogenating metal, used alone or as a mixture, and a support comprising at least one Nu-10 zeolite and at least one silica-alumina, said process being carried out at a temperature in the range 150° C. to 500° C., at a pressure in the range 0.1 MPa to 15 MPa, at an hourly space velocity in the range 0.1 to 10 h⁻¹ and in the presence of a total quantity of hydrogen mixed with the feed such that the hydrogen/feed ratio is in the range 70 to 2000 Nm³/m³ of feed.

### FIELD OF THE INVENTION

The search for new sources of renewable energy for the production of fuels constitutes a major challenge. The demand for middle distillate bases, i.e. for cuts which can be incorporated into the kerosene and gas oil pool, is greatly increasing, particularly in Europe. Using such new resources is a means of responding to this high demand while at the same time considering environmental issues, inter alia.

Within the various “alternative” pathways, middle distillate bases produced from a paraffinic feed obtained from a feed derived from renewable resources and in particular from vegetable oils or animal fats, unrefined or having undergone a prior treatment, as well as mixtures of such feeds, have particularly interesting properties. In effect, said feeds obtained from renewable resources contain triglyceride or ester or free fatty acid type chemical structures, the structure and length of the hydrocarbon feed thereof being compatible with the hydrocarbons present in the middle distillates. Said feeds obtained from renewable resources produce paraffinic feeds which are free of sulphur-containing and aromatic compounds following hydrotreatment.

Patent application EP 1 681 337 A describes the transformation of such feeds by decarboxylation in order to form paraffins with one fewer carbon atoms compared with the starting chemical structures. The advantage of this pathway as described in that patent consists in limiting the hydrogen consumption required. In contrast, the yields of gas oil bases are reduced. The catalysts used are metallic catalysts.

U.S. Pat. No. 4,992,605 and U.S. Pat. No. 5,705,722 describe processes for the production of bases for the gas oil pool produced from the direct transformation of vegetable oils (rape, palm, soya, sunflower) or from lignocellulosic biomass into saturated hydrocarbons after hydrotreatment or hydrorefining of those products alone.

The liquid effluent obtained from such hydrotreatment processes is essentially constituted by n-paraffins which may have cold properties which are insufficient for incorporation into a gas oil and/or kerosene pool. In order to improve the cold properties of that hydrotreated liquid effluent, a hydroisomerization step is necessary in order to transform the n-paraffins into branched paraffins with better cold properties. In addition, for a given number of carbon atoms, the cold properties of a paraffin generally tend to improve with the degree of isomerization of said paraffin. By way of example, for paraffins with 14 carbon atoms, the fusion temperature of n-tetradecane is 6° C., the fusion temperatures of 2-methyltridecane and 3-methyl tridecane are respectively −26° C. and −37° C. and the fusion temperature of 2,3-dimethyl dodecane is −51° C. Thus, it is also desirable to form multi-branched isomers during hydroisomerization. This hydroisomerization step is carried out on a bifunctional catalyst having both a hydrodehydrogenating function and a Bronsted acid function. Depending on the degree of incorporation and the cold properties envisaged for the final fuel, it may be necessary to carry out very intense hydroisomerization of the effluent.

This hydroisomerization step is generally accompanied by the production of cracking products which are too light to be incorporated into a gas oil and/or kerosene pool. The result, then, is a loss of yield, which it is desirable to minimize.

Patent applications EP 2 138 553 and EP 2 138 552 describe a process for the treatment of a feed obtained from a renewable resource comprising a hydrotreatment, an optional gas/liquid separation, optionally followed by elimination of nitrogen-containing compounds, and a hydroisomerization in the presence of a catalyst comprising at least one metal from group VIII and/or at least one metal from group VIB and at least one mono-dimensional 10 MR zeolite molecular sieve, preferably selected from molecular sieves of the structure type TON such as Nu-10, EUO selected from EU-1 and ZSM-50 alone or as a mixture, or the molecular sieves ZSM-48, ZBM-30, IZM-1, COK-7, EU-2 and EU-11. Said processes can be used to obtain high yields of gas oil bases.

Research carried out by the Applicant has led to the discovery that, surprisingly, the use of a catalyst comprising at least one Nu-10 zeolite and at least one silica-alumina in a process for the hydroconversion of a paraffinic feed produced from renewable resources can be used to limit the production of light cracked products which cannot be incorporated into a gas oil and/or kerosene pool while favouring the production of multi-branched isomers, the degree of branching of the effluent obtained being characteristic of an effluent with improved cold properties compared with the starting paraffinic feed.

Thus, one aim of the present invention is to provide a process for the conversion of a paraffinic feed constituted by hydrocarbons containing in the range 9 to 25 carbon atoms and obtained from renewable resources using a catalyst comprising at least one Nu-10 zeolite and at least one silica-alumina, said catalyst being highly selective in the hydroisomerization of said paraffins and allowing both limitation of the production of light cracked products and promotion of the production of multi-branched isomers.

### BRIEF DESCRIPTION OF THE DRAWINGS

FIG. 1—shows the change in the temperature as a function of the conversion for the three catalysts.

FIG. 2—shows the change in the overall yield for isomerization.

FIG. 3—shows the change in the yield of multi-branched isomers.

### AIM OF THE INVENTION

The present invention concerns a continuous process for the conversion of a paraffinic feed produced from renewable resources into middle distillate bases, gas oil and/or kerosene.

In particular, in one aspect, the present invention provides a process for the conversion of a paraffinic feed constituted by hydrocarbons containing in the range 9 to 25 carbon atoms, said paraffinic feed being produced from renewable resources, to the exclusion of paraffinic feeds obtained by a process employing a step for upgrading by the Fischer-Tropsch pathway, said process employing a catalyst comprising at least one hydrodehydrogenating metal selected from the group formed by metals from group VIB and from group VIII of the periodic classification of the elements, used alone or as a mixture, and a support comprising at least one Nu-10 zeolite and at least one silica-alumina, said process operating at a temperature in the range 150° C. to 500° C., at a pressure in the range 0.1 MPa to 15 MPa, at an hourly space velocity in the range 0.1 to 10 h⁻¹ and in the presence of a total quantity of hydrogen mixed with the feed such that the hydrogen/feed ratio is in the range 70 to 2000 Nm³/m³ of feed.

One aim of the invention is to provide a process for the conversion of a paraffinic feed produced from renewable resources for producing middle distillate bases, in particular a kerosene base and/or a gas oil base, while limiting the production of light products which cannot be incorporated into said bases.

In another aspect, the invention aims to improve the degree of branching by hydroisomerization of the paraffinic feed employed and produced from renewable resources, the degree of branching being adjusted so as to obtain properties, in particular cold properties, for the middle distillate bases which are compatible with specifications in force for middle distillates.

### SUMMARY OF THE INVENTION

The invention pertains to a process for the conversion of a paraffinic feed constituted by hydrocarbons containing in the range 9 to 25 carbon atoms, said paraffinic feed being produced from renewable resources, to the exclusion of paraffinic feeds obtained by a process employing a step for upgrading by the Fischer-Tropsch pathway, said process employing a catalyst comprising at least one hydrodehydrogenating metal selected from the group formed by metals from group VIB and from group VIII of the periodic classification of the elements, used alone or as a mixture, and a support comprising at least one Nu-10 zeolite and at least one silica-alumina, said process operating at a temperature in the range 150° C. to 500° C., at a pressure in the range 0.1 MPa to 15 MPa, at an hourly space velocity in the range 0.1 to 10 h⁻¹ and in the presence of a total quantity of hydrogen mixed with the feed such that the hydrogen/feed ratio is in the range 70 to 2000 Nm³/m³ of feed.

### DESCRIPTION OF THE INVENTION

### The Feed

In accordance with the invention, said paraffinic feed constituted by hydrocarbons containing in the range 9 to 25 carbon atoms used in the process of the invention is produced from renewable resources.

Preferably, said paraffinic feed is constituted by hydrocarbons containing in the range 10 to 25 carbon atoms, preferably in the range 10 to 22.

The quantity of paraffins in said feed employed in the process of the invention is advantageously more than 90% by weight, preferably more than 95% by weight, more preferably more than 98% by weight.

Preferably, said paraffinic feed is produced from renewable resources selected from vegetable oils, oils from algae or algals, fish oils and fats of vegetable or animal origin, or mixtures of such feeds.

According to the invention, said feed used in the process of the invention is a paraffinic feed produced from renewable resources, to the exclusion of paraffinic feeds obtained by a process employing a step for upgrading by the Fischer-Tropsch pathway. Hence, the paraffinic feeds obtained using the Fischer-Tropsch process from a synthesis gas (CO+H₂) produced from renewable resources using the BTL (biomass to liquid) process, are excluded from the feeds used in the process of the invention.

Said vegetable oils may advantageously be unrefined or completely or partially refined, and obtained from plants selected from rape, sunflower, soya, palm, olive, coconut, coprah, castor, cotton, peanut oils, linseed oil and crambe and all oils obtained, for example, from sunflower or rape by genetic modification or hybridization, this list not being limiting. Said animal fats are advantageously selected from lard and fats composed of residues from the food industry or obtained from catering industries. Frying oils, various animal oils such as fish oils, tallow or suet may also be used.

The renewable resources from which the paraffinic feed used in the process of the invention is produced essentially contain triglyceride type chemical structures which the skilled person will also know as fatty acid triesters, as well as free fatty acids the fatty chains of which contain in the range 9 to 25 carbon atoms.

The structure and length of the hydrocarbon chain of the latter is compatible with the hydrocarbons present in the gas oil and the kerosene, i.e. the middle distillates cut. A fatty acid triester is thus composed of three fatty acid chains. These fatty acid chains in the form of a triester or in the form of free fatty acids have a number of unsaturated bonds per chain, also known as the number of carbon-carbon double bonds per chain, generally in the range 0 to 3, but which may be higher, in particular for oils obtained from algae which generally have 5 to 6 unsaturated bonds per chain.

The molecules present in said renewable resources used in the present invention thus have a number of unsaturated bonds, expressed per molecule of triglyceride, which advantageously is in the range 0 to 18. In these feeds, the degree of unsaturation, expressed as the number of unsaturated bonds per fatty hydrocarbon chain, is advantageously in the range 0 to 6.

The renewable resources generally also comprise various impurities, in particular heteroatoms such as nitrogen. The nitrogen contents in the vegetable oils are generally in the range of approximately 1 ppm to 100 ppm by weight, depending on their nature. They may be as high as 1% by weight on particular feeds.

Said paraffinic feed used in the process of the invention is advantageously produced from renewable resources using processes which are known to the skilled person. One possible pathway is the catalytic transformation of said renewable resources into deoxygenated paraffinic effluent in the presence of hydrogen, and in particular hydrotreatment.

Preferably, said paraffinic feed is produced by hydrotreatment of said renewable resources. These processes for the hydrotreatment of renewable resources are already well known and are described in a number of patents. As an example, said paraffinic feed used in the process of the invention may advantageously be produced, preferably by hydrotreatment then by gas/liquid separation, from said renewable resources as described in patent FR 2 910 483 or in patent FR 2 950 895.

Preferably, said paraffinic feed is produced by hydrotreatment of said renewable resources in the presence of a fixed bed catalyst, said catalyst comprising a hydrodehydrogenating function and an amorphous support, at a temperature in the range 200° C. to 450° C., at a pressure in the range 1 MPa to 10 MPa, at an hourly space velocity in the range 0.1 h⁻¹ to 10 h⁻¹ and in the presence of a total quantity of hydrogen mixed with the feed such that the hydrogen/feed ratio is in the range 150 to 750 Nm³ of hydrogen/m³ of feed.

The catalyst used in said hydrotreatment step is a conventional catalyst preferably comprising at least one metal from group VIII and/or group VIB and at least one support selected from the group formed by alumina, silica, silica-aluminas, magnesia, clays and mixtures of at least two of these minerals. This support may also comprise other compounds, for example oxides selected from the group formed by boron oxide, zirconia, titanium oxide and phosphoric anhydride.

### The Catalyst

The process of the invention is a process for the conversion of said paraffinic feed produced from renewable resources using a catalyst comprising at least one hydrodehydrogenating metal selected from the group formed by metals from group VIB and from group VIII of the periodic classification of the elements, used alone or as a mixture, and a support comprising at least one Nu-10 zeolite and at least one silica-alumina. Preferably, said process is a hydroisomerization process.

The catalyst used in the process of the invention is advantageously bifunctional in type, i.e. it has a hydrodehydrogenating function and a hydroisomerization function. Preferably, the elements from group VIII are selected from noble metals and non-noble metals from group VIII, preferably from iron, cobalt, nickel, ruthenium, rhodium, palladium, osmium, iridium and platinum, used alone or as a mixture, and preferably from cobalt, nickel, platinum and palladium, used alone or as a mixture.

In the case in which the elements from group VIII are selected from noble metals from group VIII, the elements from group VIII are advantageously selected from platinum and palladium, used alone or as a mixture. In this case, said elements are used in their reduced form.

In the case in which the elements from group VIII are selected from non-noble metals from group VIII, the elements from group VIII are advantageously selected from cobalt and nickel, used alone or as a mixture. Preferably, the elements from group VIB are selected from tungsten and molybdenum, used alone or as a mixture. In the case in which the hydrogenating function comprises an element from group VIII and an element from group VIB, the following metal associations are preferred: nickel-molybdenum, cobalt-molybdenum, iron-molybdenum, iron-tungsten, nickel-tungsten, cobalt-tungsten, and highly preferably: nickel-molybdenum, cobalt-molybdenum, nickel-tungsten. It is also possible to use associations of three metals such as, for example, nickel-cobalt-molybdenum. When a combination of metals from group VI and group VIII is used, the catalyst is then preferably used in a sulphurized form.

In the case in which said catalyst comprises at least one noble metal from group VIII, the quantity of noble metal in said catalyst is advantageously in the range 0.01% to 5% by weight, preferably in the range 0.1% to 4% by weight and more preferably in the range 0.1% to 2% by weight with respect to the total mass of said catalyst.

In a preferred embodiment, said catalyst may also comprise tin in addition to said noble metal(s), the quantity of tin preferably being in the range 0.1% to 0.5% by weight with respect to the total catalyst mass.

In the case in which the catalyst comprises at least one metal from group VIB in combination with at least one non-noble metal from group VIII, the quantity of metal from group VIB is advantageously in the range 5% to 40% by weight of oxide with respect to the total mass of said catalyst, preferably in the range 10% to 35% by weight of oxide and highly preferably in the range 15% to 30% by weight of oxide, and the quantity of non-noble metal from group VIII is advantageously in the range 0.5% to 10% by weight of oxide with respect to the total mass of said catalyst, preferably in the range 1% to 8% by weight of oxide and highly preferably in the range 1.5% to 6% by weight of oxide.

In accordance with the invention, said catalyst comprises a support comprising at least one Nu-10 zeolite and at least one silica-alumina. In a preferred embodiment, the support for the catalyst used in the process of the invention is constituted by a Nu-10 zeolite and a silica-alumina.

Nu-10 zeolite is a ono-dimensional 10 MR crystalline microporous solid with structure type TON. The X ray diffraction table for Nu-10 zeolite and a synthesis protocol are described in patent EP 0 077 624 B1. Said Nu-10 zeolite has a chemical composition, expressed in moles, defined by the following general formula: 0.5 to 1.5 R₂₀: Y₂O₃: at least 20 XO₂: 0 to 4000 H₂O, in which R represents a monovalent cation or (1/n) of a cation with valency n, Y represents at least one element selected from aluminium, iron, chromium, vanadium, molybdenum, arsenic, antimony, manganese, gallium and boron, and X is aluminium and/or germanium.

Preferably, the Nu-10 zeolite is in the aluminosilicate form, i.e. the element Y is constituted by aluminium, and the element X is constituted by silicon.

Preferably, the molar ratio of the number of silicon atoms to the number of aluminium atoms, Si/Al, is less than 200, preferably less than 150, highly preferably less than 120.

Said Nu-10 zeolite in the composition of the catalyst support used in the process of the invention is advantageously at least in part, preferably practically completely in the acid form, i.e. in the acid form (H⁺).

To this end, when the cation R is inorganic, the zeolite is advantageously exchanged with at least one treatment using a solution of at least one ammonium salt so as to obtain the ammonium form of the Nu-10 zeolite which, once calcined, results in the acid (H⁺) form of said zeolite. This exchange step may be carried out at any step in the preparation of the catalyst. When R is a nitrogen-containing molecule, the acid form of said zeolite may be obtained by calcining; without carrying out a prior exchange step. This calcining step may be carried out at any step in the preparation of the catalyst.

The silica-aluminas used as a support for said catalyst are non-microporous solids constituted by an intimate combination of silica and alumina. Silica-aluminas can be obtained in the complete range of compositions from 0 to 100% Al₂O₃. In addition to the overall chemical composition, the degree of association of silicon and aluminium, as well as the textural properties of the solid are strongly dependent on the method of synthesis. Various synthesis protocols may be used to prepare a silica-alumina. The modes of synthesis vary as a function of the original state of the reagents employed. Aluminic and/or silicic reagents may be preformed to a greater or lesser extent, i.e., depending on the modes of synthesis, the alumina reagent will be either a solution of a metal salt which is a primary reagent or a gel which is a reagent which it is possible to qualify as a “preform”. One type of silica-alumina is synthesized by impregnation of an alumina using a preformed silica precursor (silica gel). Thus, the core of this silica-alumina is very rich in alumina, while the surface is rich in silica (W. Daniell, U. Schubert, R. Glöckler, A. Meyer, K. Noweck, H. Knözinger, Applied Catalysis A: general, 196, 147 (2000)). Another type of silica-alumina may be prepared using a sequenced method. In this case, only the silicic reagent is preformed (“silica hydrogel”) and the aluminic reagent is an aqueous solution of an aluminium salt. More precisely, the sequenced method consists of preparing the preformed silicic reagent then causing the aluminium salt to precipitate in contact with the freshly prepared silica hydrogel. A silica hydrogel may be prepared by acidification of sodium silicate with a mineral acid (sulphuric acid). A dilute aluminium salt is then added to this hydrogel (P. K. Sinhamahapatra, D. K. Sharma, R. P. Mehrotra, J. Appl. Chem. Biotechnol., 28, 740 (1978)). Another type of silica-alumina may be obtained by the co-gelling method, in which the metallic precursors are added simultaneously. Thus, the reagents present are both solutions of metallic salts. Co-gelling consists of precipitation in a single step, i.e. co-precipitation, of an aqueous solution of silicon and an aqueous solution of aluminium in the pH range where the two precursors precipitate. Any silica-alumina known to the skilled person may be suitable for the invention.

In accordance with a preferred embodiment, the silica-alumina used as a support for said catalyst contains a quantity of more than 5% by weight and less than or equal to 95% by weight of silica, preferably in the range 10% to 80% by weight, preferably a silica content of more than 20% by weight and less than 80% by weight and still more preferably more than 25% by weight and less than 75% by weight, the silica content is advantageously in the range 10% to 50% by weight. Said preferred silica-alumina advantageously has the following textural characteristics:
a mean pore diameter, measured by mercury porosimetry, in the range 20 to 140 Å;
a total pore volume, measured by mercury porosimetry, in the range 0.1 mL/g to 0.5 mL/g;
a total pore volume, measured by nitrogen porosimetry, in the range 0.1 mL/g to 0.5 mL/g;
a BET specific surface area in the range 100 to 550 m²/g;
a pore volume, measured by mercury porosimetry, included in pores with a diameter of more than 140 Å, of less than 0.1 mL/g;
a pore volume, measured by mercury porosimetry, included in pores with a diameter of more than 500 Å, of less than 0.1 mL/g;
an X ray diffraction pattern which contains at least the principal characteristic peaks of at least one of the transition aluminas included in the group composed of alpha, rho, chi, eta, gamma, kappa, theta and delta aluminas.

Preferably, said silica-alumina contains:
a quantity of cationic impurities (for example Na⁺) of less than 0.1% by weight, preferably less than 0.05% by weight and still more preferably less than 0.025% by weight. The term “quantity of cationic impurities” means the total quantity of alkalis and alkaline-earths;
a quantity of anionic impurities (for example SO₄²⁻, Cl⁻) of less than 1% by weight, preferably less than 0.5% by weight and still more preferably less than 0.1% by weight.

In one embodiment, the catalyst used in the process of the invention may advantageously contain a binder.

Said binder may advantageously be amorphous or crystalline. Preferably, said binder is advantageously selected from the group formed by alumina, silica, clays, titanium oxide, boron oxide and zirconia, used alone or as a mixture. Aluminates may also be selected. Preferably, said binder for the support is alumina. Preferably, said binder for the support is a matrix containing alumina in any of its forms which are known to the skilled person such as, for example, alpha, gamma, eta or delta type aluminas. Said aluminas differ in their specific surface area and their pore volume. Said support binder is preferably in the form of beads, grains or extrudates.

Preferably, said catalyst comprises 5% to 98% by weight of binder, highly preferably 10% to 95% by weight and still more preferably 20% to 95% by weight with respect to the total mass of said catalyst.

In the case in which said catalyst contains a binder, the catalyst comprises a total quantity of Nu-10 zeolite and silica-alumina which is advantageously in the range 1.5% to 94.5%, preferably in the range 10% to 80%, more preferably in the range 20% to 70% by weight with respect to the total mass of said catalyst. Preferably, the quantity by weight of the Nu-10 zeolite is less than the quantity by weight of the silica-alumina.

In accordance with another embodiment, the catalyst used in the process of the invention does not contain binder. In this case, said catalyst advantageously comprises a total Nu-10 zeolite and silica-alumina content of at least 50%, preferably at least 57%, highly preferably at least 64% by weight with respect to the total mass of said catalyst.

Preferably, the quantity by weight of Nu-10 zeolite is less than the quantity by weight of silica-alumina.

In accordance with a preferred embodiment, the support is constituted by a silica-alumina and a Nu-10 zeolite.

In another preferred embodiment, the support is constituted by a silica-alumina, a Nu-10 zeolite and a binder.

The support comprising at least one Nu-10 zeolite and at least one silica-alumina is advantageously prepared from solids prepared as described above using any of the methods which are well known to the skilled person.

The Nu-10 zeolite may advantageously be introduced using any method which is known to the skilled person and at any stage in the preparation of the support or catalyst.

A preferred process for the preparation of the catalyst of the present invention advantageously comprises the steps described below.

In accordance with a preferred mode of preparation, the Nu-10 zeolite may advantageously be introduced during synthesis of the precursors of the silica-alumina. Without being limiting in any way, the Nu-10 zeolite may, for example, be in the form of a powder, ground powder, suspension, suspension which has undergone a deagglomeration treatment. Thus, for example, the Nu-10 zeolite may advantageously be taken up into suspension which may or may not be acidulated, to a concentration adjusted to the final envisaged zeolite content on the support. This suspension, routinely known as a slip, is then mixed with the precursors of the silica-alumina at any stage of its synthesis, as described above.

In accordance with another preferred mode of preparation, the Nu-10 zeolite and the silica-alumina may advantageously also be introduced during shaping of the support with an optional at least one binder. Without being limiting in any way, the Nu-10 zeolite and the silica-alumina may advantageously be in the form of a powder, ground powder, suspension, or suspension which has undergone a deagglomeration treatment.

Advantageously, Nu-10 zeolite and silica-alumina in the powder form are mixed, then the mixture is shaped.

Shaping may be carried out using any technique which is known to the skilled person such as, for example, extrusion, pelletization, shaping into beads using a rotary or drum granulator, oil drop, oil up coagulation, or bowl granulator.

The supports obtained thereby are shaped into the form of grains of different shapes and dimensions. They are generally used in the form of cylindrical or polylobed extrudates such as bilobes, trilobes or polylobes, with a straight or twisted shape, but they may also be fabricated and employed in the form of crushed powders, tablets, rings, beads or wheels.

After shaping, the catalyst support used in the process of the present invention may advantageously undergo various heat treatments. The support may initially undergo a drying step. Said drying step is advantageously carried out using any technique which is known to the skilled person.

Preferably, drying is carried out in a stream of air. Said drying may also advantageously be carried out in a stream of any oxidizing, reducing or inert gas. Preferably, drying is advantageously carried out between 50° C. and 180° C., preferably between 60° C. and 150° C. and highly preferably between 80° C. and 130° C.

Said support, which may optionally be dried, then preferably undergoes a calcining step.

Said calcining step is advantageously carried out in the presence of molecular oxygen, for example by flushing with air, at a temperature which is advantageously more than 200° C. and less than or equal to 1100° C. Said calcining step may advantageously be carried out in a flushed bed, trickle bed or in a static atmosphere. As an example, the furnace used may be a rotary furnace or a vertical furnace with radial flushed layers. Preferably, said calcining step is carried out between more than one hour at 200° C. to less than one hour at 1100° C. Calcining may advantageously be carried out in the presence of steam and/or in the presence of an acidic or basic vapour. As an example, calcining may be carried out under a partial pressure of ammonia.

Post-calcining treatments may optionally be carried out in order to improve the properties of the support, for example the textural properties.

The support comprising the Nu-10 zeolite, the silica-alumina and optional binder may then optionally undergo a hydrothermal treatment in a confined atmosphere. The term “hydrothermal treatment in a confined atmosphere” means a treatment by passage through an autoclave in the presence of water at a temperature which is above ambient temperature.

During this hydrothermal treatment, the support can advantageously be treated. Thus, the support can advantageously be impregnated prior to passage through the autoclave, autoclaving being carried out either in the vapour phase or in the liquid phase, this vapour or liquid phase of the autoclave possibly being acidic or non-acidic. This impregnation prior to autoclaving may advantageously be acidic, or it may not. This impregnation prior to autoclaving may advantageously be carried out dry or by immersing the support in an aqueous acidic solution. The term “dry impregnation” means bringing the support into contact with a volume of solution which is less than or equal to the total pore volume of the support. Preferably, dry impregnation is carried out.

The autoclave is preferably a rotary basket autoclave such as that defined in patent application EP-A-0 387 109.

The temperature during autoclaving may be in the range 100° C. to 250° C. for a period of time in the range 30 minutes to 3 hours.

The hydrodehydrogenating function may advantageously be introduced at any step of the preparation of the catalyst, highly preferably after shaping the support constituted by the Nu-10 zeolite, the silica-alumina and optional binder. Shaping is advantageously followed by calcining; the hydrodehydrogenating function may also advantageously be introduced before or after this calcining. The preparation is generally finished by calcining at a temperature of 250° C. to 600° C. Another preferred method of the present invention advantageously consists of shaping the support after mixing it, then passing the paste obtained through a die to form extrudates. The hydrodehydrogenating function may advantageously then be introduced in part only or in its totality at the moment of mixing. It may also advantageously be introduced onto the calcined support using one or more ion exchange operations.

Preferably, the support is impregnated using an aqueous solution. Impregnation of the support is preferably carried out using the “dry” impregnation method which is well known to the skilled person. Impregnation may advantageously be carried out in a single step using a solution containing all of the constituent elements of the final catalyst.

The hydrodehydrogenating function may advantageously be introduced using one or more operations for impregnating the shaped and calcined support, using a solution containing at least one precursor of at least one oxide of at least one metal selected from the group formed by metals from group VIII and metals from group VIB, the precursor(s) of at least one oxide of at least one metal from group VIII preferably being introduced after those of group VIB or at the same time thereas, if the catalyst contains at least one metal from group VIB and at least one metal from group VIII.

In the case in which the catalyst advantageously contains at least one element from group VIB, for example molybdenum, it is, for example, possible to impregnate the catalyst with a solution containing at least one element from group VIB, then to dry and calcine. Impregnation of molybdenum may advantageously be facilitated by adding phosphoric acid to the solutions of ammonium paramolybdate, which means that phosphorus can also be introduced in a manner so as to promote the catalytic activity.

The following elements: boron and/or silicon and/or phosphorus may be introduced into the catalyst at any stage of the preparation and using any technique which is known to the skilled person.

One preferred method in accordance with the invention consists of depositing the selected promoter element or elements, for example the boron-silicon pairing, onto the support which may or may not have been shaped and is preferably calcined. To this end, an aqueous solution of at least one boron salt such as ammonium biborate or ammonium pentaborate is prepared in an alkaline medium and in the presence of hydrogen peroxide and “dry” impregnation is carried out, in which the volume of the pores of the support is filled with the solution containing boron, for example. In the case in which silicon is also deposited, for example, a solution of a silicone type silicon compound or a silicone oil emulsion is used, for example.

The promoter element or elements selected from the group formed by silicon, boron and phosphorus may advantageously be introduced using one or more impregnation operations, using an excess of solution, onto the calcined precursor. The source of boron may advantageously be boric acid, preferably orthoboric acid, H₃BO₃, ammonium biborate or pentaborate, boron oxide, or boric esters. The boron may, for example, be introduced in the form of a mixture of boric acid, hydrogen peroxide and a basic organic compound containing nitrogen such as ammonia, primary or secondary amines, cyclic amines, compounds from the pyridine family and quinolines and compounds from the pyrrole family. The boron may, for example, be introduced using a boric acid solution in a water/alcohol mixture.

The preferred source of phosphorus is orthophosphoric acid, H₃PO₄, but its salts and esters such as ammonium phosphates are also suitable. The phosphorus may, for example, be introduced in the form of a mixture of phosphoric acid and a basic organic compound containing nitrogen such as ammonia, primary and secondary amines, cyclic amines, compounds from the pyridine family and quinolines and compounds from the pyrrole family.

Many sources of silicon may advantageously be employed. Thus, it is possible to use ethyl orthosilicate Si(OEt)₄, siloxanes, polysiloxanes, silicones, silicone emulsions, halogen silicates such as ammonium fluorosilicate (NH₄)₂SiF₆ or sodium fluorosilicate Na₂SiF₆. Silicomolybdic acid and its salts, or silicotungstic acid and its salts may also advantageously be employed. The silicon may, for example, be added by impregnating ethyl silicate in solution in a water/alcohol mixture. The silicon may, for example, be added by impregnation of a silicone or silicic acid type silicon compound suspended in water.

The noble metals from group VIII of the catalyst of the present invention may advantageously be present completely or partially in the metal and/or oxide form.

The sources of noble elements from group VIII which may advantageously be used are well known to the skilled person. For noble metals, halides are used, for example chlorides, nitrates, acids such as chloroplatinic acid, hydroxides, oxychlorides such as ammoniated ruthenium oxychloride. It is also advantageously possible to use cationic complexes such as ammonium salts.

The catalysts obtained thereby are shaped into the form of grains of varying shapes and dimensions. They are generally used in the form of cylindrical or polylobed extrudates such as bilobes, trilobes or polylobes with a straight or twisted shape, but they may also be fabricated and employed in the form of crushed powders, tablets, rings, beads or wheels. Preferably, the catalysts used in the process of the invention are in the shape of spheres or extrudates. However, it is advantageous for the catalyst to be in the form of extrudates with a diameter in the range 0.5 to 5 mm, more particularly in the range 0.7 to 2.5 mm. The shapes are cylindrical (they may or may not be hollow), twisted cylinders, multilobes (2, 3, 4 or 5 lobes, for example), or rings. The cylindrical shape is advantageously and preferably used, but any other shape may advantageously be used.

The shaped catalyst of the invention advantageously generally has a crush strength of at least 70 N/cm, preferably 100 N/cm or higher.

In the case in which the catalyst used in the process of the invention comprises at least one noble metal, the noble metal contained in said catalyst must be reduced. The metal is advantageously reduced by a treatment in hydrogen at a temperature in the range 150° C. to 650° C. and a total pressure in the range 0.1 to 25 MPa. As an example, a reduction consists in a stage at 150° C. for two hours, then a temperature ramp-up to 450° C. at a rate of 1° C./min, then a stage lasting two hours at 450° C.; throughout this reduction step, the hydrogen flow rate is 1000 normal m³ of hydrogen per m³ of catalyst and the total pressure is kept constant at 0.1 MPa. Any ex situ reduction method may advantageously be envisaged.

In the case in which the catalyst used in the process of the invention comprises at least one metal from group VIB in combination with at least one non-noble metal from group VIII, the metals are preferably used in their sulphurized form. The catalyst may be sulphurized in situ or ex situ using any method which is known to the skilled person.

### Conversion Process

The paraffinic feed constituted by hydrocarbons containing in the range 9 to 25 carbon atoms and produced from renewable resources is brought into contact with said catalyst in the presence of hydrogen at temperatures and operating pressures which advantageously mean that conversion can be carried out, preferably hydroisomerization, which can be used to obtain the envisaged cold properties.

In accordance with the invention, said process is carried out at a temperature in the range 150° C. to 500° C., at a pressure in the range 0.1 MPa to 15 MPa, at an hourly space velocity in the range 0.1 to 10 h⁻¹ and in the presence of a total quantity of hydrogen mixed with the feed such that the hydrogen/feed ratio is in the range 70 to 2000 Nm³/m³ of feed.

Preferably, said process is carried out at a temperature in the range 150° C. to 450° C., highly preferably in the range 200° C. to 450° C., at a pressure in the range 0.2 to 15 MPa, preferably in the range 0.5 to 10 MPa and highly preferably in the range 1 to 9 MPa, at an hourly space velocity which is advantageously in the range 0.2 to 7 h⁻¹, preferably in the range 0.5 to 5 h⁻¹, and in the presence of a total quantity of hydrogen mixed with the feed such that the hydrogen/feed ratio is in the range 100 to 1500 normal m³ of hydrogen per m³ of feed, preferably in the range 150 to 1500 normal m³ of hydrogen per m³ of feed.

Preferably, at least a portion, and preferably all of the effluent obtained from the conversion process of the invention undergoes one or more separation steps so as to recover a cut boiling at a temperature in the range 150° C. to 370° C. The aim of this step is to separate the gases from the liquid, and in particular to recover gases which are rich in hydrogen which may also contain light gases such as the C₁-C₄ cut, and at least one cut boiling at a temperature in the range 150° C. to 370° C. corresponding to a gas oil base and/or a kerosene base, preferably a kerosene base.

### EXAMPLES

### Example 1

### Preparation of a Pt—SiAl Hydroisomerization Catalyst C1 (not in Accordance with the Invention)

The catalyst C1 was a catalyst containing a noble metal, platinum, and at least one silica-alumina. It was a commercial silica-alumina in the form of extrudates, supplied by AXENS. This silica-alumina contained 35% by weight of silica and 35% by weight of alumina, according to the results obtained by X ray fluorescence. Said silica-alumina had the following characteristics:
a BET specific surface area of 225 m²/g;
a total pore volume, measured by nitrogen porosimetry, of 0.4 mL/g;
a pore volume, measured by mercury porosimetry, included in pores with a diameter of more than 140 Å, of 0.02 mL/g;
a pore volume, measured by mercury porosimetry, included in pores with a diameter of more than 500 Å, of 0.01 mL/g;
a mean pore diameter, measured by mercury porosimetry, of 72 Å;
a sodium content, measured by atomic absorption, of less than 0.025% by weight.

The silica-alumina extrudates were first ground then screened in order to recover a powder with a granulometry in the range 355 to 500 microns. Platinum was then deposited onto the powder by dry impregnation using an aqueous solution of a Keller complex, Pt(NH₃)₄Cl₂. After oven drying overnight at 110° C., the powder was dry impregnated with an aqueous solution of Pt(NH₃)₄Cl₂, left to mature, typically for 24 hours at ambient temperature, then calcined in a flushed bed in a flow of dry air fixed at 2 normal litres per hour per gram of solid, at successive temperature stages of 150° C. for 1 hour, 250° C. for 1 hour, 350° C. for one hour and finally 520° C. for two hours. The quantity of platinum by weight, measured by XRF on the finished catalyst after calcining, was 0.1% by weight.

### Example 2

### Preparation of Pt—Nu-10 Hydroisomerization Catalyst C2 (not in Accordance with the Invention)

Catalyst C2 was a catalyst containing a noble metal, platinum and a Nu-10 zeolite. The Nu-10 zeolite was synthesised using the protocol described in Example 1 of patent EP 0 077 624 B 1, starting from a reaction mixture having the following molar composition:
60 SiO₂; 0.8 Al₂O₃; 8.7K₂O; 18 DAH; 2470 H₂O
   in which DAH corresponds to 1,6 diaminohexane.

The as-synthesised zeolite then underwent calcining in a thin layer in a muffle furnace at 200° C. for two hours (temperature ramp-up 2° C./min), then at 550° C. for twelve hours (temperature ramp-up 1° C./min). In order to obtain the zeolite in its ammonium form, the calcined zeolite was then exchanged with an aqueous solution of 10M ammonium nitrate (10 mL of solution per gram of solid) with stirring and under reflux for 4 hours. The solid was then rinsed with distilled water and recovered by centrifuging and oven dried in a thin layer overnight. The exchange, rinsing and drying operations were carried out three times. In order to obtain the zeolite in its acid (H⁺) form, the powder was then calcined in a flushed bed in a flow of dry air fixed at 2 normal litres per hour per gram of solid, with successive temperature stages of 150° C. for one hour, 250° C. for one hour, 350° C. for one hour and finally 550° C. for four hours. The zeolite obtained had a Si/Al atomic ratio (determined by X ray fluorescence) of 31 and a potassium content, measured by atomic absorption, of 0.042% by weight.

Platinum was then deposited on the powder by dry impregnation using an aqueous solution of a Keller complex, Pt(NH₃)₄Cl₂. After oven drying overnight at 110° C., the zeolite was dry impregnated with an aqueous solution of Pt(NH₃)₄Cl₂, left to mature typically for 24 hours at ambient temperature, then calcined in a flushed bed in a flow of dry air (fixed at 2 normal litres per hour per gram of solid) at successive temperature stages of 150° C. (for 1 hour), 250° C. (for 1 hour), 350° C. (for one hour) and finally 520° C. (for two hours). The quantity of platinum by weight, measured by XRF on the catalyst after calcining, was 0.4% by weight.

Finally, catalyst C2 was shaped by pelletizing the powder on a hydraulic press then grinding and screening the pellets obtained in order to recover a powder with a granulometry in the range 355 to 500 microns.

### Example 3

### Preparation of Pt—Nu-10/SiAl Hydroisomerization Catalyst C3 (in Accordance with the Invention)

Catalyst C3 was a catalyst containing a noble metal, platinum, Nu-10 zeolite and silica-alumina. This catalyst was prepared by mixing catalyst C1 and catalyst C2 in a ball mill. After mixing in the ball mill, the mixture obtained was shaped by pelletizing on a hydraulic press then grinding and screening the pellets obtained in order to recover a powder with a granulometry in the range 355 to 500 microns. The quantities of catalyst C1 and catalyst C2 were adjusted so as to obtain a catalyst C3 with an overall composition: 0.12% by weight Pt/5.97% by weight Nu-10/93.90% by weight silica-alumina.

### Example 4

### Hydroisomerisation of n-Hexadecane

A synthetic paraffinic feed composed of 80% by weight of n-heptane (Carlo Erba, 99% by weight) and 20% by weight of n-hexadecane (Halternann, 99% by weight) was hydroisomerized on various hydroisomerization catalysts in a flushed bed in a high flow rate test unit using the protocol described in the literature (F. Marques Mota et al., Prep. Pap. Am. Chem. Soc., Div. Pet. Chem., 2012, 57(1), 145). It was verified that under the test operating conditions, the solvent n-heptane was not converted with the catalysts C1, C2 and C3. The hydroisomerized hydrocarbon effluent was analysed using an in-line chromatography system installed on the unit. The catalytic performances of the catalysts were evaluated from the chromatographic results. Before the catalytic test, each catalyst underwent a reduction step in a flow of hydrogen under the following operating conditions:
total pressure: 0.1 MPa;
hydrogen flow rate: 2000 normal litres per hour per litre of catalyst;
temperature rise from ambient temperature to 450° C. at 5° C./minute;
one hour stage at 450° C.

After the reduction step, the pressure and the temperature were adjusted to the desired values and the feed was injected. The operating conditions for the n-hexadecane hydroisomerization reaction were as follows:
total pressure: 0.5 MPa;
HSV (volume of feed/volume of catalyst/hour): 11 h⁻¹;
hydrogen/feed ratio: 1800 normal litres/litre;
temperature: varying.

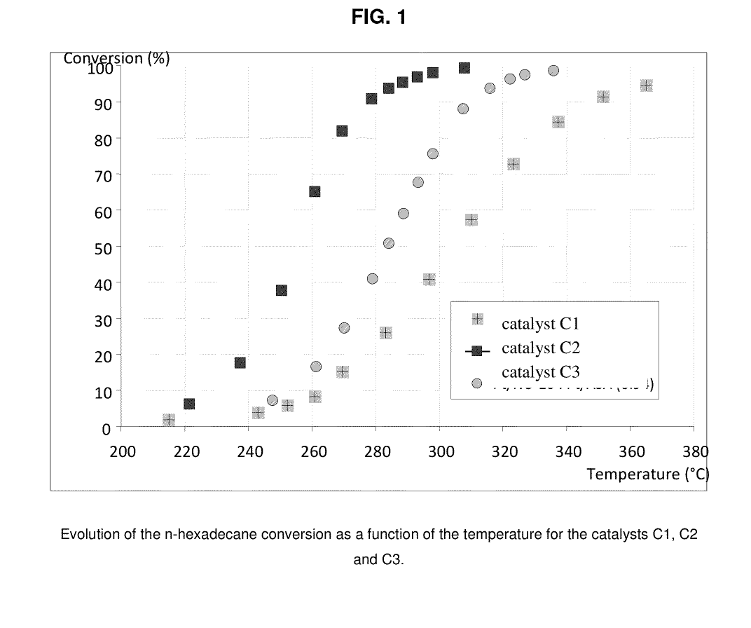

For each catalyst, different test temperatures were used in order to vary the degree of conversion of the n-hexadecane. For the catalyst C1, the temperature was thus varied between 210° C. and 370° C., for catalyst C2 the temperature was varied between 220° C. and 310° C. and for the catalyst C3, the temperature was varied between 240° C. and 340° C. FIG. 1 reports the change in the temperature as a function of the conversion for the three catalysts. As expected, the activity of catalyst C3 was intermediate between that of catalyst C1 and that of catalyst C2.

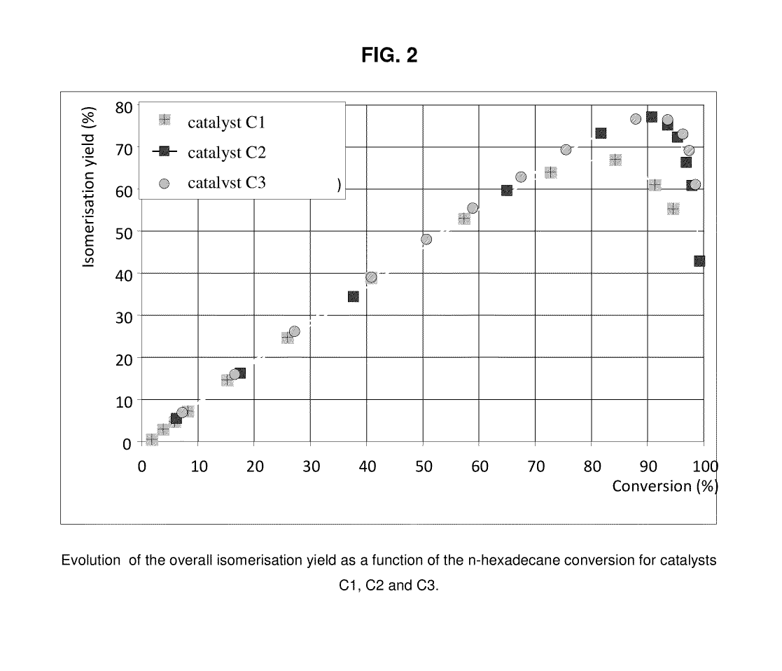

FIG. 2 reports the change in the overall yield for isomerization (mono-branched and multi-branched isomers of n-hexadecane) as a function of the conversion of n-hexadecane for the three catalysts. As expected, catalyst C2 based on Nu-10 zeolite could produce higher isomerization yields than catalyst C1, based on silica-alumina, for conversions of higher than 70%. Surprisingly, the behaviour of catalyst C3, based on Nu-10 zeolite and silica-alumina, was not intermediate between catalysts C1 and C2, but produced isomerization yields comparable to those observed with catalyst C2.

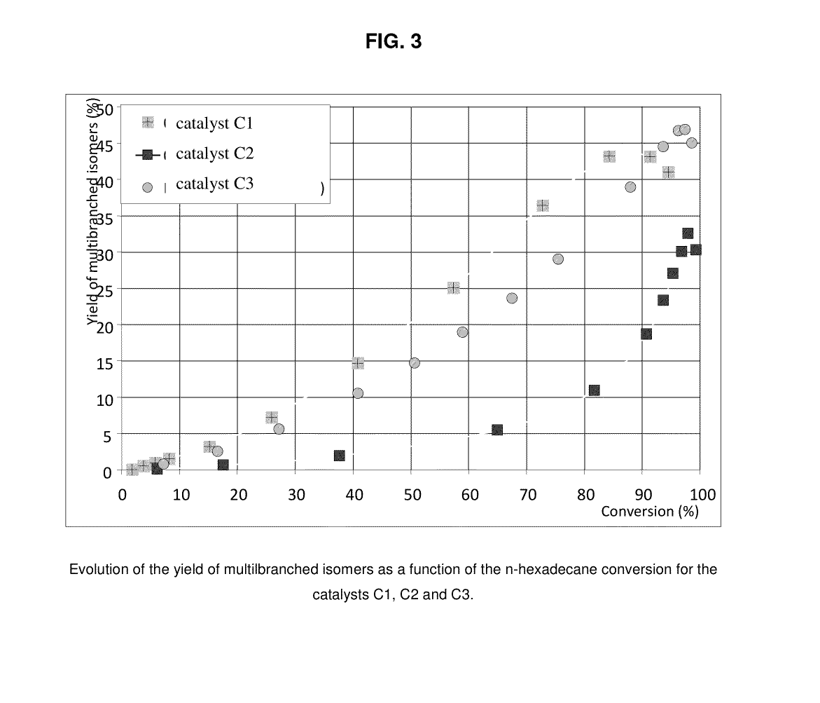

FIG. 3 reports the change in the yield of multi-branched isomers as a function of the conversion of n-hexadecane for the three catalysts. Over the conversion range being studied, catalyst C1, based on silica-alumina, could produce yields of multi-branched isomers which were higher than catalyst C2, based on Nu-10 zeolite. Surprisingly, the behaviour of catalyst C3 was not intermediate between C1 and C2, but could produce the highest yields of multi-branched isomers for conversions of more than 90%.

Hence, catalyst C3 can be used on the one hand to obtain isomerization yields comparable to those obtained for catalyst C2 and higher than those obtained with catalyst C1 for conversions of more than 70% and on the other hand can be used to obtain the highest yields of multi-branched isomers for conversions of more than 90%. Table 1 thus reports the performances of catalysts C1, C2 and C3 for maximum overall isomerization yields (yieldₘₐₓ iso-C₁₆ in Table 1) obtained for each of the catalysts.

Compared with C1, C3 can be used to obtain a maximum overall yield in isomerization which is higher by 10 points.

Compared with C2, for the same overall maximum isomerization yield of 77%, C3 can be used to obtain a yield of multi-branched isomers which is higher by 20 points (iso-C₁₆ multi-branched yield in Table 1).

Further, the use of the catalyst of the invention C3 means that a lower cracking yield can be obtained (yield, cracking in Table 1) than catalysts C1 and C2, which means that the production of light cracked products can be limited.

TABLE 1
Performances of catalysts C1, C2 and C3 for maximum overall
isomerization yields obtained for each of the catalysts

| Catalyst | C1 | C2 | C3 |
|---|---|---|---|
| Yieldₘₐₓ, iso-C₁₆ (%) | 67 | 77 | 77 |
| Yield, iso-C₁₆ mono-branched (%) | 24 | 58 | 38 |
| Yield, iso-C₁₆ multi-branched (%) | 43 | 19 | 39 |
| Yield, cracking (%) | 17 | 14 | 11 |

The entire disclosures of all applications, patents and publications, cited herein and of corresponding French application Ser. No. 13/52527, filed Mar. 21, 2013 are incorporated by reference herein.

1. A process for the conversion of a paraffinic feed constituted by hydrocarbons containing in the range 9 to 25 carbon atoms, said paraffinic feed being produced from renewable resources, to the exclusion of paraffinic feeds obtained by a process employing a step for upgrading by the Fischer-Tropsch pathway, said process employing a catalyst comprising at least one hydrodehydrogenating metal selected from the group formed by metals from group VIB and from group VIII of the periodic classification of the elements, used alone or as a mixture, and a support comprising at least one Nu-10 zeolite and at least one silica-alumina, said process operating at a temperature in the range 150° C. to 500° C., at a pressure in the range 0.1 MPa to 15 MPa, at an hourly space velocity in the range 0.1 to 10 h⁻¹ and in the presence of a total quantity of hydrogen mixed with the feed such that the hydrogen/feed ratio is in the range 70 to 2000 Nm³/m³ of feed.

2. The process as claimed in claim 1, in which said paraffinic feed is constituted by hydrocarbons containing in the range 10 to 22 carbon atoms.

3. The process as claimed in claim 1, in which said paraffinic feed is produced from renewable resources selected from vegetable oils, oils from algae or algals, fish oils and fats of animal or vegetable origin, or mixtures of such feeds.

4. The process as claimed in claim 1, in which said process in accordance with the invention is a hydroisomerization process.

5. The process as claimed in claim 1, in which the elements from group VIII are selected from cobalt, nickel, platinum and palladium, used alone or as a mixture.

6. The process as claimed in claim 5, in which the quantity of noble metal of said catalyst is in the range 0.01% to 5% by weight with respect to the total mass of said catalyst.

7. The process as claimed in claim 1, in which the elements from group VIB are selected from tungsten and molybdenum, used alone or as a mixture.

8. The process as claimed in claim 1, in which the quantity of metal from group VIB is in the range 5% to 40% by weight of oxide with respect to the total mass of said catalyst, and the quantity of non-noble metal from group VIII is in the range 0.5% to 10% by weight of oxide with respect to the total mass of said catalyst.

9. The process as claimed in claim 1, in which the silica-alumina used in the support for said catalyst contains a quantity of more than 5% by weight and less than or equal to 95% by weight of silica and has the following textural characteristics:
a mean pore diameter, measured by mercury porosimetry, in the range 20 to 140 Å;
a total pore volume, measured by mercury porosimetry, in the range 0.1 mL/g to 0.5 mL/g;
a total pore volume, measured by nitrogen porosimetry, in the range 0.1 mL/g to 0.5 mL/g;
a BET specific surface area in the range 100 to 550 m²/g;
a pore volume, measured by mercury porosimetry, included in pores with a diameter of more than 140 Å, of less than 0.1 mL/g;
a pore volume, measured by mercury porosimetry, included in pores with a diameter of more than 500 Å, of less than 0.1 mL/g;
an X ray diffraction pattern which contains at least the principal characteristic peaks of at least one of the transition aluminas included in the group composed of alpha, rho, chi, eta, gamma, kappa, theta and delta aluminas.

10. The process as claimed in claim 1, in which said catalyst contains a binder, said binder being selected from the group formed by alumina, silica, clays, titanium oxide, boron oxide and zirconia, used alone or as a mixture.

11. The process as claimed in claim 10, in which said catalyst comprises 5% to 98% by weight of binder with respect to the total mass of said catalyst.

12. The process as claimed in claim 10, in which said catalyst comprises a total quantity of Nu-10 zeolite and silica-alumina in the range 1.5% to 94.5% by weight with respect to the total mass of said catalyst, the quantity by weight of Nu-10 zeolite being less than the content by weight of silica-alumina.

13. The process as claimed in claim 1, in which said catalyst does not contain binder.

14. The process as claimed in claim 13, in which said catalyst comprises a total quantity of Nu-10 zeolite and silica-alumina of at least 50% by weight with respect to the total mass of said catalyst.

15. The process as claimed in claim 1, in which said process is carried out at a temperature in the range 150° C. to 450° C., at a pressure in the range 0.2 to 15 MPa, at an hourly space velocity in the range 0.2 to 7 h⁻¹ and in the presence of a total quantity of hydrogen mixed with the feed such that the hydrogen/feed ratio is in the range 100 to 1500 normal m³ of hydrogen per m³ of feed.
